(19) United States
(12) Patent Application Publication
(10) Pub. No.: US 2007/0016236 A1
(43) Pub. Date: Jan. 18, 2007

(54) Balanced ultrasonic curved blade

(75) Inventor: Jean Beaupre, Cincinnati, OH (US)

Correspondence Address: JAMES C. EAVES JR.;GREENEBAUM DOLL & MCDONALD PLLC, 3500 NATIONAL CITY TOWER, 101 SOUTH FIFTH STREET, LOUISVILLE, KY, 40202, US

(73) Assignee: Crescendo Technologies, LLC

(21) Appl. No.: 11/411,731

(22) Filed: Apr. 26, 2006

Related U.S. Application Data
(60) Provisional application No. 60/700,079, filed on Jul. 18, 2005.
(60) Provisional application No. 60/717,288, filed on Sep. 15, 2005.

(51) Int. Cl. A61B 17/32

(52) U.S. Cl. 606/169

(57) ABSTRACT

Methods and devices that provide reduced transverse motion in a curved ultrasonic blade and/or ultrasonic surgical instrument with functional asymmetries. An ultrasonic blade in accordance with embodiments of the present invention includes a curved functional portion of an ultrasonic blade, wherein the center of mass of the curved functional portion lies on the mid-line of a waveguide delivering ultrasonic energy to the blade. Balancing in accordance with embodiments of the present invention, using placement of the center of mass of the curved portion of the blade appropriately, provides blade balance in a proximal portion of the blade, without reduction of mass and inherent stress increase proximal to the end-effector.

### RELATED APPLICATIONS

This application is related to, and claims the benefit of, U.S. Provisional Patent Applications Ser. No. 60/700,079 filed Jul. 18, 2005, and Ser. No. 60/717,288 filed Sep. 15, 2005, which are hereby incorporated herein by reference.

### FIELD OF THE INVENTION

The present invention relates, in general, to ultrasonic devices and, more particularly, to methods and devices that provide curved blades with reduced undesired transverse motion.

### BACKGROUND OF THE INVENTION

The fields of ultrasonics and stress wave propagation encompass applications ranging from non-destructive testing in materials science, to beer packaging in high-volume manufacturing. Diagnostic ultrasound uses low-intensity energy in the 0.1-to-20-MHz region to determine pathological conditions or states by imaging. Therapeutic ultrasound produces a desired bio-effect, and can be divided further into two regimes, one in the region of 20 kHz to 200 kHz, sometimes called low-frequency ultrasound, and the other in the region from 0.2 to 10 MHz, where the wavelengths are relatively small, so focused ultrasound can be used for therapy. At high intensities of energy, this application is referred to as HIFU for High Intensity Focused Ultrasound.

Examples of therapeutic ultrasound applications include HIFU for tumor ablation and lithotripsy, phacoemulsification, thrombolysis, liposuction, neural surgery and the use of ultrasonic scalpels for cutting and coagulation. In low-frequency ultrasound, direct contact of an ultrasonically active end-effector or surgical instrument delivers ultrasonic energy to tissue, creating bio-effects. Specifically, the instrument produces heat to coagulate and cut tissue, and cavitation to help dissect tissue planes. Other bio-effects include: ablation, accelerated bone healing and increased skin permeability for transdermal drug delivery.

Ultrasonic medical devices are used for the safe and effective treatment of many medical conditions. Ultrasonic surgical instruments are advantageous because they may be used to cut and/or coagulate organic tissue using energy, in the form of mechanical vibrations, transmitted to a surgical end-effector at ultrasonic frequencies. Ultrasonic vibrations, when transmitted to organic tissue at suitable energy levels and using a suitable end-effector, may be used to cut, dissect, or cauterize tissue.

Ultrasonic vibration is induced in the surgical end-effector by, for example, electrically exciting a transducer which may be constructed of one or more piezoelectric or magnetostrictive elements in the instrument hand piece. Vibrations generated by the transducer section are transmitted to the surgical end-effector via an ultrasonic waveguide extending from the transducer section to the surgical end-effector. The waveguide/end-effector combinations are typically designed to resonate at the same frequency as the transducer. Therefore, when an end-effector is attached to a transducer the overall system frequency is still the same frequency as the transducer itself.

At the tip of the end-effector, ultrasonic energy is delivered to tissue to produce several effects. Effects include the basic gross conversion of mechanical energy to both frictional heat at the blade-tissue interface, and bulk heating due to viscoelastic losses within the tissue. In addition, there may be the ultrasonically induced mechanical mechanisms of cavitation, microstreaming, jet formation, and other mechanisms.

Ultrasonic surgical instruments utilizing solid core technology are particularly advantageous because of the amount of ultrasonic energy that may be transmitted from the ultrasonic transducer through a solid waveguide to the active portion of the end-effector, typically designated as a blade. Such instruments are particularly suited for use in minimally invasive procedures, such as endoscopic or laparoscopic procedures, wherein the end-effector is passed through a trocar to reach the surgical site.

Solid core ultrasonic surgical instruments may be divided into two types, single element end-effector devices and multiple-element end-effector. Single element end-effector devices include instruments such as scalpels, and ball coagulators. See, for example, U.S. Pat. No. 5,263,957. Multiple element end-effectors include those illustrated in devices such as ultrasonic shears, for example, those disclosed in U.S. Pat. Nos. 5,322,055 and 5,893,835 provide an improved ultrasonic surgical instrument for cutting/coagulating tissue, particularly loose and unsupported tissue. The ultrasonic blade in a multiple-element end-effector is employed in conjunction with a clamp for applying a compressive or biasing force to the tissue. Clamping the tissue against the blade provides faster and better controlled coagulation and cutting of the tissue.

In an ultrasonic device running at resonance in primarily a longitudinal mode, the longitudinal ultrasonic motion, d, behaves as a simple sinusoid at the resonant frequency as given by:
d=A sin(ω·t)

where: ω=the radian frequency, which equals (2·π) multiplied by the cyclic frequency, f; t is time; and A=the zero-to-peak amplitude.

The longitudinal excursion is defined as the peak-to-peak amplitude, which is twice the amplitude of the sine wave, mathematically expressed as 2·A.

An ultrasonic waveguide and blade in perfect balance over its entire length will vibrate longitudinally according to this simple harmonic motion. Unfortunately, ultrasonic blades are not typically in perfect balance. For example, blades useful for medical applications may incorporate curves or features that cause blade imbalances.

### SUMMARY OF THE INVENTION

The present invention is directed to methods and devices that provide reduced transverse motion in a curved ultrasonic blade. An ultrasonic blade in accordance with embodiments of the present invention includes a curved functional portion of an ultrasonic blade, wherein the center of mass of the curved functional portion lies on the mid-line of a waveguide delivering ultrasonic energy to the blade. Balancing in accordance with embodiments of the present invention, using placement of the center of mass of the curved portion of the blade appropriately, provides blade balance in a proximal portion of the blade, without reduction of mass and inherent stress increase proximal to the end-effector.

Embodiments of ultrasonic surgical devices in accordance with the present invention include an elongated waveguide configured to transmit ultrasonic energy. The elongated waveguide has a center-line extending through the center of mass. An end-effector is provided at the distal end of the waveguide, and includes a curved portion having a positive curvature. The positive curvature of the curved portion produces an offset of the center of mass of the curved portion. An anti-curved portion is positioned between the elongated waveguide and the curved portion, the anti-curve having a negative curvature, the negative curvature configured to correct the offset of the center of mass of the curved portion, thereby substantially balancing the ultrasonic surgical device.

Other embodiments have the anti-curved portion locating the center of mass of the curved portion about the center-line such that the non-longitudinal excursion in the waveguide proximal to the end-effector is below 5% of the primary vibration excursion. Further embodiments include a clamp arm configured to opposably clamp tissue against the curved portion, wherein the clamp arm is actuatably movable from an open position to a clamped position.

Methods of balancing ultrasonic systems in accordance with embodiments of the present invention involve determining a center-line that extends through the center of mass of a first portion of an ultrasonic system. A center of mass of a second portion of the ultrasonic system is determined, the second portion comprising an asymmetry. The center of mass of the second portion is located about the center-line of the first portion using a curved portion of the ultrasonic system, the curved portion positioned between the first portion and the second portion.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

The features of the invention may be set forth with particularity in the appended claims. The invention itself, however, both as to organization and methods of operation, together with further objects and advantages thereof, may best be understood by reference to the following description, taken in conjunction with the accompanying drawings in which:

FIG. 1 is a perspective view of an ultrasonic blade having the center of mass of the curved portion placed in accordance with an embodiment of the present invention;

FIG. 2 is a top view of the ultrasonic blade having the center of mass of the curved portion placed in accordance with embodiments of the present invention as illustrated in FIG. 1;

FIG. 3 is a side view of the ultrasonic blade having the center of mass of the curved portion placed in accordance with embodiments of the present invention as illustrated in FIG. 1;

FIG. 4 is a side view of an ultrasonic blade having the center of mass of the curved portion placed in accordance with embodiments of the present invention, the blade incorporated into a clamping instrument with the clamp arm open;

FIG. 5 is a side view of an ultrasonic blade having the center of mass of the curved portion placed in accordance with embodiments of the present invention, the blade incorporated into a clamping instrument with the clamp arm closed;

FIG. 6 is a perspective view of an ultrasonic blade having the center of mass of the curved portion placed in accordance with embodiments of the present invention, the blade incorporated into a clamping instrument with the clamp arm open; and

FIG. 7 is a perspective view of an ultrasonic blade having the center of mass of the curved portion placed in accordance with embodiments of the present invention, the blade incorporated into a clamping instrument with the clamp arm closed.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF THE INVENTION

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

Considerable effort has been directed at correcting imbalances inherent in curved ultrasonic blades and ultrasonic devices that are not symmetric about their longitudinal axis. Descriptions of methods to correct ultrasonic blade imbalances are described in U.S. Pat. Nos. 6,283,981; 6,328,751; 6,660,017; 6,325,811; 6,432,118; and 6,773,444, which are hereby incorporated herein by reference. Although balancing of ultrasonic blades has greatly expanded the possibilities of blade design, balancing using the methodologies described in U.S. Pat. Nos. 6,283,981; 6,328,751; and 6,660,017 require balance asymmetries proximal to the functional portion of the blade. Balancing methodologies described in U.S. Pat. Nos. 6,325,811; 6,432,118; and 6,773,444 describe the use of functional asymmetries in the end-effector that may be used for balancing.

Balancing using asymmetries proximal to the end-effector using reductions of mass inherently causes reduction in strength due to the lost mass at the balance asymmetry. Balancing using asymmetries in the end-effector, such as is described in U.S. Pat. Nos. 6,325,811; 6,432,118; and 6,773,444 require machining and alteration of blade shape in the functional portion of the blade. Balancing in accordance with the present invention, using placement of the center of mass of a curved portion about the centerline of a waveguide portion to reduce transverse motion in the waveguide portion, provides blade balance in a proximal portion of the blade without the reduction of mass and inherent stress increase proximal to the end-effector.

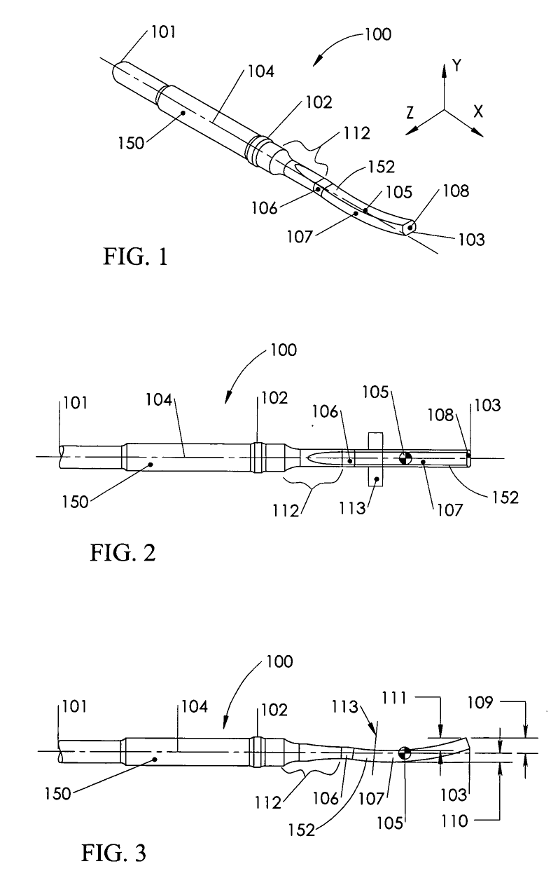

Referring now to FIG. 1, a perspective view of an ultrasonic surgical instrument 100 is illustrated, including a waveguide 150 and a blade 152. In accordance with embodiments of the present invention, the ultrasonic surgical instrument 100 includes a curved treatment portion 107 for use in medical procedures to, for example, dissect or cut living organic tissue. A distal flat working surface 108 is illustrated as terminating the curved treatment portion 107, and may be used for spot coagulation, plane dissection, or other surgical procedure.

A center of mass 105 of the curved treatment portion 107 is located on a central axis 104 of the waveguide 150. The central axis 104 may be defined as the center-line of a circularly symmetric blade extending along the longitudinal direction, or a line extending in the primary vibrational-mode direction and passing through the center of mass, for blades that are not circularly symmetric. The center of mass 105 is illustrated in FIG. 1 as about 0.01 inches laterally from the central axis 104, and may be about 0.0003 inches laterally from the central axis 104.

The ultrasonic surgical instrument 100 is illustrated in FIG. 1 as extending from a proximal anti-node 101 to a distal anti-node 103, with a distal node 102 approximately half way between the proximal anti-node 101 and the distal anti-node 103. An amplifier 112 may be included to amplify the excursion of the blade. The amplifier 112 may provide about a multiple of 2 amplification (about a one-half reduction of diameter.) An anti-curve 106 may be positioned between the distal node 102 and the curved treatment portion 107, to position the center of mass 105 at or near the central axis 104, thereby providing reduction of transverse motion in the waveguide 150 in accordance with the present invention. The anti-curve 106 and the curved treatment portion 107 may be used in combination as a functional portion of the ultrasonic surgical instrument 100 in particular embodiments of the present invention. In other embodiments, the anti-curve 106 may be provided proximal to the functional portion of the ultrasonic surgical instrument 100. In the particular embodiment illustrated in FIGS. 1 through 3, the anti-curve 106 and the curved treatment portion 107 are both part of the functional portion of the blade. The anti-curve 106 is illustrated in FIG. 1 as about 0.053 inches to about 0.061 inches in length, and may be about 0.015 λ to about 0.018 λ in some alternate embodiments.

In the particular embodiment illustrated in FIG. 1, the cross sections of the curved treatment portion 107 and the waveguide 150 are symmetrical. The deflection of the curved treatment portion 107 of the ultrasonic surgical instrument 100 is substantial, in order to create an out and around shape to aid in medical surgical procedures, and to allow passage through a trocar or endoscopic surgical port (not shown.) For example, the curvature of the curved treatment portion 107 is illustrated as having a continuous or varying arc of about 15 to 30 degrees that may be accomplished, for example, using a radius of curvature of about 1.2 inches over a length of about 0.6 inches. In the particular embodiment illustrated in FIGS. 1 through 3, the radius of curvature is illustrated as 1.192 inches through an arc of about 27.22 degrees. The radius of curvature for top and bottom surfaces of the curved treatment portion 107 may be different. For example, the bottom surface of the curved treatment portion 107 may have a radius of curvature of about 1.22 inches, while the top surface of the curved treatment portion 107 may have a radius of curvature of about 1.163 inches.

The ultrasonic surgical instrument 100 is preferably made from a solid core shaft constructed of material which propagates ultrasonic energy, such as a titanium alloy (i.e., Ti-6Al-4V) or an aluminum alloy. It will be recognized that the ultrasonic surgical instrument 100 may be fabricated from any other suitable material. It is also contemplated that the ultrasonic surgical instrument 100 may have a surface treatment to improve the delivery of energy and desired tissue effect. For example, the ultrasonic surgical instrument 100 may be micro-finished, coated, plated, etched, grit-blasted, roughened or scored to enhance coagulation and cutting of tissue and/or reduce adherence of tissue and blood. Additionally, the ultrasonic surgical instrument 100 may be sharpened or shaped to enhance its characteristics. For example, a portion of the curved treatment portion 107 may be shaped, sharpened, or have some other desired shape.

FIGS. 2 and 3 are top and side views respectively of the ultrasonic surgical instrument 100 illustrated in FIG. 1, illustrating the three dimensional positioning of the center of mass 105 relative to the central axis 104. In the particular example illustrated in FIGS. 1 through 3, the anti-curve 106 is illustrated as angling the curved treatment portion 107 about 6 degrees to about 12 degrees, and more particularly about 8.13 degrees, to position the center of mass 105 about the central axis 104, thereby reducing undesired transverse motion in the waveguide 150.

The ultrasonic surgical instrument 100 having the curved treatment portion 107 incorporated mechanical asymmetries that naturally have a tendency to include tip excursion in at least two, and possibly all three axes, x, y, and z of a three-dimensional right-handed coordinate system. If not balanced properly, excursions other than longitudinal will reflect a moment or force back to the transducer, causing inefficiencies and/or loss of lock to the longitudinal drive frequency, and possibly failure and/or fracture. For example, the curved treatment portion 107 may be described as having a positive curvature in the x-y plane. This curvature will cause excursions in at least both the x and y directions when activated.

It is possible to balance forces and/or moments caused by non-longitudinal tip excursion of a functional asymmetry, such as the curved treatment portion 107, by placing the center of mass of the curved treatment portion 107 about the center-line of the ultrasonic system in accordance with the present invention. It is desirable to balance a system below 15% non-longitudinal excursion proximal to the functional asymmetry, and it is preferable to balance below 5% non-longitudinal excursion proximal to the functional asymmetry. One method of locating the center of mass about the center line uses an anti-curve, such as the anti-curve 106.

A normalized non-longitudinal excursion percentage in an ultrasonic blade may be calculated by taking the magnitude of the excursion in the non-longitudinal direction, and dividing that magnitude by the magnitude of the maximum vibration excursion in the longitudinal direction (also called the primary vibration excursion), and then multiplying the dividend by one hundred. Primary tip vibration excursion is the magnitude of the major axis of the ellipse or ellipsoid created by a point on the distal most end, designated the terminal end, of curved treatment portion 107 when the ultrasonic surgical instrument 100 is activated. The primary tip vibration excursion and the primary vibration excursion may be equivalent or different, depending on the relationship between the longitudinal motion direction and the direction of the major axis of the ellipse or ellipsoid.

FIGS. 2 and 3 illustrate a cross-section plane 113, normal to the tangent of the longitudinal axis of the curved treatment portion 107, in which the blade 152 is symmetric about both the vertical and horizontal axes in the illustrated embodiment. The cross section of the curved treatment portion 107 at the cross-section plane 113 is illustrated as substantially rectangular, with dimensions about 0.057 inches height by about 0.085 inches width. In some alternate embodiments, the cross section of the curved treatment portion 107 at the cross-section plane 113 may be about 0.016 λ height by about 0.024 λ width. The curved treatment portion 107 is illustrated as about 0.545 inches to about 0.572 inches in length, and about 0.156 λ to about 0.164 λ in some alternate embodiments.

FIG. 3 illustrates a tip deflection 109 of about 0.070 inches of the edge of the curved treatment portion 107 relative to the center line 104. In some alternate embodiments the tip deflection 109 may be about 0.020 λ, for example. A curve deflection 110 of about 0.040 inches of the bottom of the curved treatment portion 107 relative to the center line 104 is also illustrated. In some alternate embodiments the curve deflection 110 may be about 0.011 λ, for example. A curve depth 111 of about 0.060 inches of the top of the curved treatment portion 107 relative to the center line 104 is also illustrated. In some alternate embodiments the curve depth 111 may be about 0.018 λ, for example.

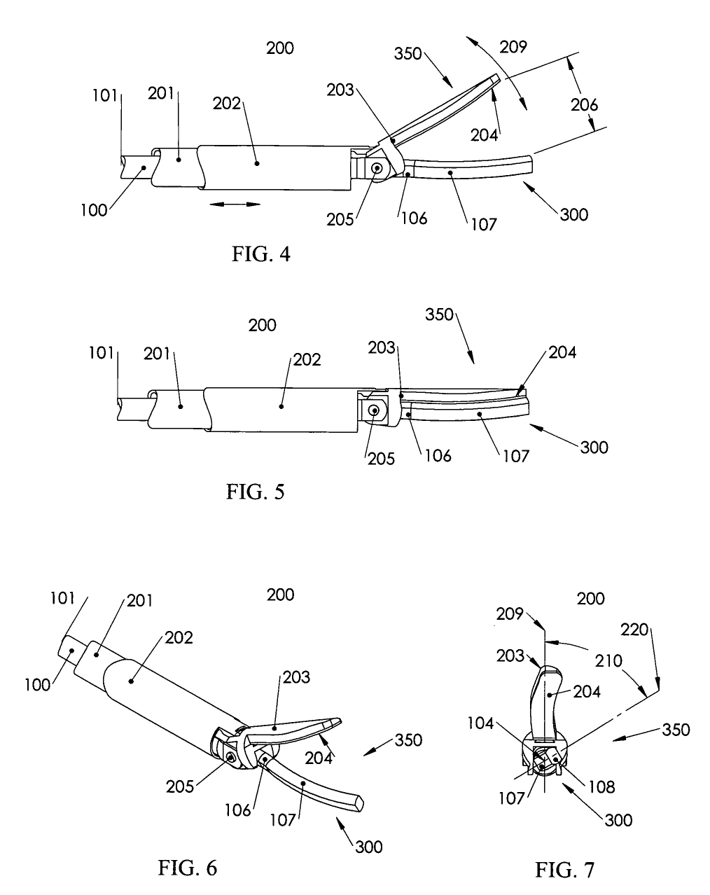

FIGS. 4 through 7 illustrate a blade 300, balanced in accordance with the present invention, in combination with a clamp arm 203, the combination designated as an end-effector 350. For purposes of illustration, and not limitation, the ultrasonic surgical instrument 100 illustrated in FIGS. 1 through 3 is illustrated in FIGS. 4 through 7 as the blade 300 of the end-effector 350. The end-effector 350 illustrated in FIGS. 4 through 7 includes the blade 300 that is configured to operate in clamping cooperation with a clamp arm 203. The clamp arm 203 includes a clamp pad 204 configured to apply pressure against the blade 300 in order to cut and/or coagulate tissue disposed between the clamp arm 203 and the blade 300.

The proximal anti-node 101 is illustrated in FIGS. 4 through 7 as a cut-away from the proximal portion of the waveguide, including the transducer and actuating mechanisms (not shown). As is known in the art, the waveguide may include any number of half-wave sections, and may be ultrasonically activated by “Langevin” piezo-electric transducers, magnetostrictive transducers, or using other methodology of causing reciprocal resonant motion of the ultrasonic system. Suitable transducers and actuating mechanisms are further described in U.S. Pat. Nos. 6,283,981; 6,328,751; 6,660,017; 6,325,811; 6,432,118; and 6,773,444, previously incorporated by reference.

The ultrasonic surgical instrument 100 is illustrated in FIGS. 4 through 7 the lumen of an inner tube 201 and an outer tube 202. The inner tube 201 is illustrated as pivotally retaining the clamp arm 203 using a pivot pin 205. The outer tube 202 is illustrated as rotatably coupled to the clamp arm 203, such as by using a hook and slot or other suitable hinged connection. Motion of the outer tube 202 acts through the separation between the pivot pin 205 and the coupling to the clamp arm 203 to raise and lower the clamp arm 203 against the blade 300. In the fully open position, the clamp arm 203 provides an opening 206 between the blade 300 and the clamp pad 204 to engage tissue for surgical applications. The clamp arm 203 rotates about the pivot pin 205 causing the clamp arm 203 to move in a clamp plane 209 as the outer tube 202 moves relative to the inner tube 201.

Referring now to FIG. 7, the clamp plane 209 is illustrated, in this particular embodiment, as extending through the central axis 104 of the ultrasonic surgical instrument 100. The curved treatment portion 107 of the ultrasonic surgical instrument 100 is curved along a plane 220 at an angle 210 relative to the clamp plane 209. For example, the plane 220 may be at an angle 210 of 0 degrees to 180 degrees. The plane 220 is preferably at an angle 210 of about 30 degrees to 70 degrees, and most preferably at an angle of about 50 degrees to about 70 degrees. The angle 210 may be fixed during manufacture, or may be adjustable by the operator.

Example embodiments illustrated herein include mass balancing in accordance with the present invention. Typically, symmetrical mass balance may be implemented using symmetrical cross-sections of waveguide and blade portions, thereby reducing the amount of imbalance in an ultrasonic surgical instrument. Curved blade shapes in accordance with the present invention include their center of mass centered about the central axis of the blade's waveguide. An anti-curve proximal to curve of the blade may be used to position the blade's center of mass about the waveguides central axis, thereby reducing undesirable transverse motion in the waveguide.

Curved blade portions provide for an out and around surgical technique, and allow for passage of the blade through a trocar. Embodiments of blades in accordance with the present invention may include a flat front surface that may be used as a coagulating surface. The flat front surface may alternately be modified as a cutting surface. Curved blades used in clamping instruments may incorporate non-parallel motion with respect to their clamp pad, aiding in cutting and coagulation.

The dimensions shown in the figures and the above text are for purposes of illustration and not of limitation. For example, dimensions may vary, typically up to 35% from the designated numbers, without departing from the scope of the present invention. Dimensions may be given as multiples of the wavelength λ, for example, Ti6Al4V titanium alloy may have a ½ wavelength λ=1.74 inches at 55.5 kHz. It is understood that varying a dimension of an element may require altering other dimensions in order to maintain balance in accordance with the present invention.

Each feature disclosed in this specification (including any accompanying claims, abstract, and drawings), may be replaced by alternative features having the same, equivalent or similar purpose, unless expressly stated otherwise. Thus, unless expressly stated otherwise, each feature disclosed is one example only of a generic series of equivalent or similar features.

While embodiments of the present invention have been shown and described herein, it will be obvious to those skilled in the art that such embodiments are provided as examples only. Numerous variations, changes, and substitutions will be apparent to those skilled in the art without departing from the invention. Accordingly, it is intended that the invention be limited only by the scope of the appended claims.

What is claimed is:

1. An ultrasonic blade, comprising:
a waveguide configured to transmit ultrasonic energy therethrough;
an end-effector provided at a distal end of the waveguide, the end-effector comprising a functional asymmetry and having a center of mass; and
means for balancing a non-longitudinal excursion in the waveguide proximal to the end-effector, the balancing means comprising a placement of the center of mass of the functional asymmetry about a center-line of the waveguide.

2. The blade of claim 1, wherein the functional asymmetry comprises a curve having a positive curvature, and wherein the balancing means comprises an anti-curve having a negative curvature.

3. The blade of claim 2, wherein the anti-curved portion locates the center of mass of the curved portion about the center-line such that the non-longitudinal excursion in the waveguide proximal to the end-effector is below 5% of the primary vibration excursion.

4. The blade of claim 1, further comprising a clamp arm configured to opposably clamp against the curved portion, the clamp arm actuatably movable from an open position to a clamped position.

5. The blade of claim 4, wherein motion of the blade is not parallel to the clamp arm in the clamped position at one or more contact points.

6. The Blade of claim 4, wherein the motion of the blade is parallel to the clamp arm in the clamped poition at one or more contact points.

7. The blade of claim 1, wherein the profile of said blade will not extend beyond 3 millimeters from the center-line.

8. The blade of claim 1, wherein a cross sectional profile perpendicular to the center-line does not intersect the center-line.

9. An ultrasonic surgical device, comprising:
an elongated waveguide having a center of mass and configured to transmit ultrasonic energy therethrough, the elongated waveguide comprising a center-line extending through the center of mass of the elongated waveguide;
an end-effector provided at a distal end of the waveguide, the end-effector comprising a curved portion having a positive curvature, the positive curvature of the curved portion producing an offset of the center of mass of the curved portion; and
an anti-curved portion positioned between the elongated waveguide and the curved portion, the anti-curve having a negative curvature, the negative curvature configured to correct the offset of the center of mass of the curved portion, thereby substantially balancing the ultrasonic surgical device.

10. The device of claim 9, wherein the anti-curved portion locates the center of mass of the curved portion about the center-line such that the non-longitudinal excursion in the waveguide proximal to the end-effector is below 5% of the primary vibration excursion.

11. The device of claim 9, further comprising a clamp arm configured to opposably clamp against the curved portion, the clamp arm actuatably movable from an open position to a clamped position.

12. A method of balancing ultrasonic systems, comprising:
determining a center-line that extends through the center of mass of a first portion of an ultrasonic system;
determining a center of mass of a second portion of the ultrasonic system, the second portion comprising an asymmetry; and
locating the center of mass of the second portion about the center-line of the first portion using a curved portion of the ultrasonic system, the curved portion positioned between the first portion and the second portion.
